(19) United States
(12) Patent Application Publication
(10) Pub. No.: US 2025/0222209 A1
(43) Pub. Date: Jul. 10, 2025

(54) WARMING DEVICE AND METHOD

(71) Applicant: Bayer HealthCare LLC, Whippany, NJ (US)

(72) Inventor: Christopher Andrew STOUT, Pleasanton, CA (US)

(21) Appl. No.: 18/852,811

(22) PCT Filed: Mar. 23, 2023
(86) PCT No.: PCT/US2023/016040

§ 371 Date: Sep. 30, 2024

Related U.S. Application Data
(60) Provisional application No. 63/326,613, filed on Apr. 1, 2022.

(51) Int. Cl. A61M 5/44

(52) U.S. Cl. CPC A61M 5/445; A61M 2205/3368

(57) ABSTRACT

Devices, kits, and methods for heating a drug formulation in an injection device are disclosed. An embodiment of the device includes a housing enclosure having a first housing shell and a second housing shell, and a plurality of bladders adjacent to an inner surface of the first housing shell and adjacent to an inner surface of the second housing shell. The housing enclosure is designed to receive the injection device. When an edge of the first housing shell and an edge of the second housing shell couple, the bladders contact a portion of the injection device and heat the drug formulation in the injection device. An embodiment of the kit includes this device and the injection device.

### BACKGROUND

The present embodiments are directed to a warming device and method, particularly for warming an injection device with a drug formulation. The injection of a drug formulation into a patient can be uncomfortable and even painful. One method of increasing comfort and reducing pain is to lower the viscosity of the drug formulation before injecting the drug into the patient. Lowering viscosity reduces the corresponding injection force. Thus, the patient receiving the drug formulation at a lower viscosity may feel more comfort and less pain compared to receiving the drug formulation at a higher viscosity.

Reducing viscosity is especially important for formulations with high drug concentrations, such as a formulation with a high concentration of biologics. The relationship between drug viscosity and drug concentration is logarithmic. Thus, drug formulations with higher concentrations would have an exponentially higher viscosity and higher corresponding injection force. Moreover, higher drug concentrations may lead to aggregations in the formulation that can cause injection problems that further increase pain during injection. Providing a formulation with a reduced and more uniform viscosity can help address discomfort and pain associated with higher drug concentrations and aggregations.

One way to reduce viscosity of a drug formulation and make the viscosity more uniform is to increase the drug formulation temperature. Drug viscosity may be lowered exponentially by increasing the drug formulation's temperature to lower the cohesive forces between molecules in the formulation. However, a patient may need to wait as long as 15-30 minutes for the formulation to reach room temperature after the formulation is removed from refrigeration. Furthermore, while the injection device containing the drug formulation may be heated with hot water, an oven, or a microwave, such heating processes may cause contamination, overheating of the drug formulations, and/or damage to the injection device. In addition, there are many types of injection devices, such as pre-filled syringes, autoinjectors, cartridges, and ampoules-all of which have different geometries. It may be costly and inefficient to use different heating devices or mechanisms to lower drug formulation viscosity in different injection devices having different geometries.

### SUMMARY

Devices, kits, and methods for heating a drug formulation in an injection device are disclosed. An embodiment of the device includes a housing enclosure having a first housing shell and a second housing shell, and a plurality of bladders adjacent to an inner surface of the first housing shell and adjacent to an inner surface of the second housing shell. The housing enclosure is designed to receive the injection device. When an edge of the first housing shell and an edge of the second housing shell couple, the bladders contact a portion of the injection device and heat the drug formulation in the injection device. The embodiments also comprise a kit including this injection device.

Some embodiments of a device for heating a drug formulation in an injection device comprise: a housing enclosure comprising a first housing shell having an inner surface, a second housing shell having an inner surface. When the housing enclosure is in an open configuration, an edge of the first housing shell and an edge of the second housing shell are de-coupled to provide an opening for receiving the injection device. The device may further comprise a plurality of bladders comprising: a first bladder adjacent to the inner surface of the first housing shell, and a second bladder adjacent to the inner surface of the second housing shell; and a heating element designed to provide heat to the plurality of bladders for heating the drug formulation. When the housing enclosure is in a closed configuration, the edge of the first housing shell and the edge of the second housing shell couple and form an open-ended structure, and the bladders contact a portion of the injection device for heating the drug formulation in the injection device.

The plurality of bladders may comprise enclosures for containing a fluid.

The device may further comprise a sensor for detecting a temperature of the injection device.

The sensor may comprise a thermocouple.

When the housing enclosure is in the closed configuration, the plurality of bladders may define a lumen with a star-shaped cross-sectional surface area for contacting and holding the injection device.

The plurality of bladders may be inflated and deformable to conform to the portion of the injection device.

The plurality of bladders may comprise silicone, nylon, latex, rubber, an elastomer material, or combination thereof.

The injection device may comprise an autoinjector.

The injection device may comprise a pre-filled syringe.

The device may further comprise a base attached to the housing enclosure.

The device may comprise a latch for coupling the edge of the first housing shell and the edge of the second housing shell.

The drug formulation may comprise a biologic.

A portion of the injection device not in contact with the plurality of bladders may protrude from the open-ended structure.

Some embodiments of a kit comprise: an injection device and a device for heating a drug formulation in the injection device, comprising: a housing enclosure comprising: a first housing shell having an inner surface, a second housing shell having an inner surface. When the housing enclosure is in an open configuration, an edge of the first housing shell and an edge of the second housing shell are de-coupled to provide an opening for receiving the injection device. The device may further comprise: a plurality of bladders comprising: a first bladder adjacent to the inner surface of the first housing shell, and a second bladder adjacent to the inner surface of the second housing shell; and a heating element designed to provide heat to the plurality of bladders for heating the drug formulation. When the housing enclosure is in a closed configuration: the edge of the first housing shell and the edge of the second housing shell couple and form an open-ended structure, and the bladders contact a portion of the injection device for heating the drug formulation in the injection device.

Some embodiments of a method for heating a drug formulation in an injection device using a warming device comprise: de-coupling an edge of a first housing shell of the warming device and an edge of a second housing shell of the warming device and providing an opening; receiving, via the opening, the injection device; coupling the edge of the first housing shell and the edge of the second housing shell and contacting a plurality of bladders of the warming device with a portion of the injection device; and heating, via the plurality of bladders, the injection device.

Heating, via the plurality of bladders, the injection device may comprise heating the bladders at one atmosphere to about 21-22.2° C. within five seconds to 10 minutes from storage temperature.

The method may further comprise: determining, based on an output of a sensor of the warming device, whether the temperature of the injection device has reached a desired temperature or a desired temperature range; in accordance with a determination that the temperature of the injection device has reached the desired temperature or the desired temperature range, ceasing heating the drug formulation; and in accordance with a determination that the temperature of the injection device has not reached the desired temperature or the desired temperature range, continuing heating the drug formulation.

The method may further comprise maintaining the temperature of the injection device at a desired temperature or a desired temperature range.

The plurality of bladders may be inflated, and the method may further comprise causing the plurality of bladders to conform to the portion of the injection device.

Coupling the edge of the first housing shell and the edge of the second housing shell may further comprise latching the first housing shell and the edge of the second housing shell.

### DESCRIPTION OF THE FIGURES

FIG. 1 depicts an exemplary warming device when the housing enclosure is in a closed configuration, in accordance with some embodiments.

FIG. 2 depicts an exemplary warming device when the housing enclosure is in an open configuration, in accordance with some embodiments.

FIG. 3 depicts exemplary bladders of a warming device, in accordance with some embodiments.

FIG. 4 depicts an exemplary latch of a warming device, in accordance with some embodiments.

FIGS. 5-7 depict exemplary warming devices when the housing enclosure is in an open configuration and exemplary injection devices, in accordance with some embodiments.

FIG. 8 depicts an exemplary warming device when the housing enclosure is in a closed configuration and an exemplary injection device, in accordance with some embodiments.

FIG. 9 depicts an exemplary method of heating a drug formulation, in accordance with some embodiments.

FIGS. 10-11 depict exemplary warming devices and exemplary injection devices, in accordance with some embodiments.

### DETAILED DESCRIPTION

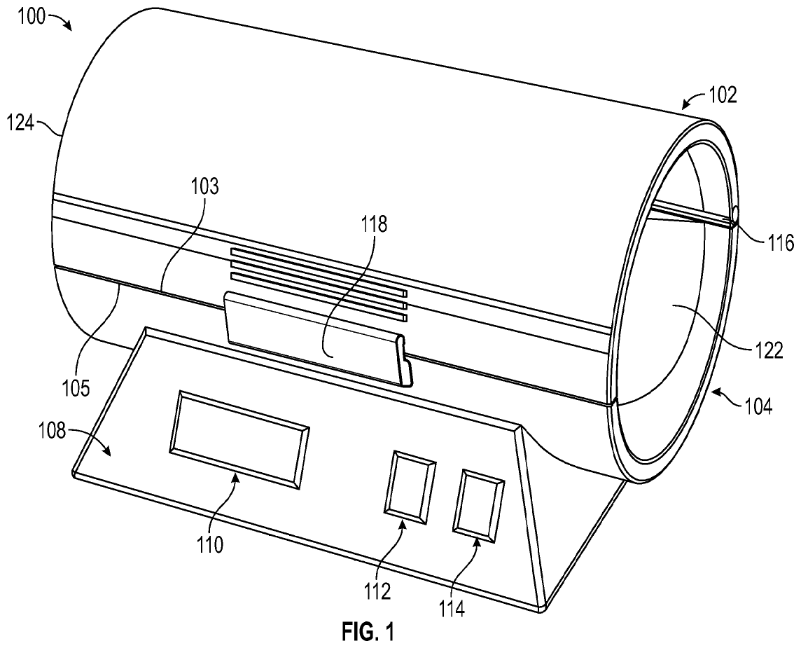

FIG. 1 depicts a perspective view of an exemplary warming device 100. The warming device 100 comprises a housing enclosure that includes a first housing shell 102 and a second housing shell 104. As depicted in FIG. 1, the housing enclosure is in the closed configuration—an edge 103 of the first housing shell 102 and an edge 105 of the second housing shell 104 are coupled via a latch 118. The warming device 100 comprises a hinge 116 that connects a second edge of the first housing shell 102 and a second edge of the second housing shell 104 and allows the first housing shell 102 to rotate about the hinge 116. The warming device 100 comprises a latch 118 on the second housing shell 104 below the edge 105.

The first housing shell 102 and the second housing shell 104 may each have a half cylinder shape. When the housing enclosure is in the closed configuration, the first housing shell 102 and the second housing shell 104 form a cylindrical tube having open ends 122 and 124. Although the first housing shell 102 and the second housing shell 104 are illustrated to form a cylinder, it should be appreciated that the housing shells may form different shapes. For example, the housing shells may form a hexagonal prism, an octagonal prism, a decagonal prism, or another shape for receiving and surrounding an injection device, as described in more detail below.

The first housing shell 102 and the second housing shell 104 may be molded or 3D-printed. The first housing shell 102 and the second housing shell 104 may comprise a thermally insulating material, such as plastic, for keeping heat away from an exterior of the warming device 100.

The warming device 100 comprises a base 108. The base 108 may comprise a half-cylinder opening to accommodate the shape of the second housing shell 104 for interfacing the two parts. The second housing shell 104 may be bolted or welded to the base 108. The base 108 may house electronic components for controlling the warming device 100.

A heat activation button 110, a warming indicator light 112, and a ready light 114 may be disposed on a side of the base 108 (for example, on a same side as latch 118). A user may push the heat activation button 110 to cause the warming device 100 to initiate heating of a drug formulation in an injection device. The warming indicator light 112 indicates a heating status of the warming device. The ready light 114 indicates whether the drug formulation in the injection device reached a desired temperature or temperature range.

The desired temperature or temperature range is typically specific to the drug formulation and may be room temperature, body temperature, or above body temperature. For example, room temperature is 18-28° C., body temperature is 35.6-37.2° C., and above body temperature is up to 40° C. These temperatures and ranges are exemplary. Other temperatures and ranges, such as a temperature range between room temperature and a temperature that would overheat the drug formulation, may be used.

Although warming device 100 is described as comprising one button and two lights, it should be appreciated that the warming device 100 may comprise additional, fewer, or different user interface elements. For example, the warming device 100 may comprise an interface for setting a desired temperature or a desired temperature range. As another example, the warming device 100 may comprise an interface for receiving information associated with a drug formulation to be heated. The information may comprise injection device type and information associated with drug formulation viscosity. The received information may be used to determine heating time. The interface for receiving information may be an input device, a barcode or a QR code scanner, a visual recognition sensor, or a RFID sensor. As another example, the warming device 100 may comprise one light designed to illuminate different colors for indicating heating of a drug formulation in an injection device and indicating when the warming device 100 finishes heating the drug formulation.

The warming device 100 may be electrically powered. For example, the warming device 100 comprises a power cord for receiving power from a power outlet. As another example, the warming device 100 is powered by one or more batteries. The one or more batteries may be rechargeable via the power from the power cord.

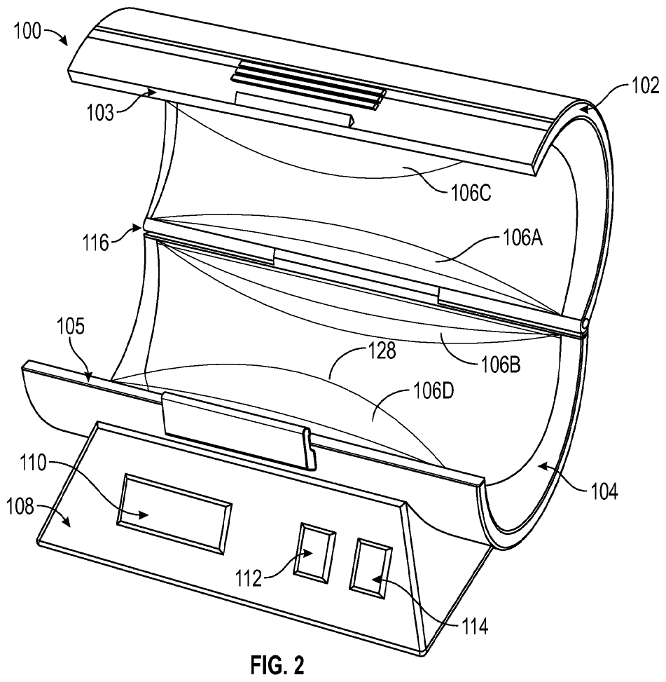

FIG. 2 depicts an exemplary warming device 100 when the housing enclosure is in an open configuration. When the housing enclosure is in the open configuration, the edge 103 of the first housing shell 102 and the edge 105 of the second housing shell 104 are de-coupled. The hinge 116 connecting the first housing shell 102 and the second housing shell 104 allows the first housing shell 102 to rotate about the hinge 116. The rotation causes the edge 103 to move away from the edge 105 to de-couple the housing shells. The de-coupling of the housing shells provides an opening for receiving an injection device (as described in more detail below).

The warming device 100 comprises a plurality of bladders. For example, the warming device 100 comprises bladders 106A-106D. It should be appreciated that the illustrated bladder configuration is exemplary. It should also be appreciated that the bladders of the warming device 100 may have different shape, lengths, widths, and/or curvatures than described. For example, the cross-section of a bladder may be triangular or rectangular. The plurality of bladders 106A-106D are inflated (by a fluid, such as a gas, a liquid, or a combination). When an injection device contacts a bladder, at least a portion of the bladder can deform and conform to the portion of the injection device in contact. The bladders 106A-106D comprise a flexible material, such as silicone, nylon, latex, rubber, an elastomer material, or any combination thereof, to allow this deformation. Each of the plurality of bladders 106A-106D provides an enclosure for containing a fluid. For example, the enclosure comprises a lumen with an aperture for receiving the fluid. The fluid can be a gas, such as air; a liquid, such as water; or a solution such as water with a thermally conductive additive, oil, or a thermally conductive fluid. The fluid can also be any combination thereof.

The warming device 100 comprises a heating element 128. The heating element 128 comprises electrical heating elements, such as heating coils, heating ribbons, and/or heating mats. The heating ribbons may be flexible heating ribbons, and the heating mats may be flexible heating mats. The heating element may be positioned in the first housing shell 102 and/or the second housing shell 104. The heating element may be positioned in the base 108. The heating element may be positioned in one or more of the bladders 106A-106D. Through thermal conduction, the heating element is designed to heat the fluid in the bladders to a specific temperature or range, such as a temperature for heating the drug formulation to a desired temperature or a desired temperature range. Through conduction, the bladders heat an injection device in the housing enclosure via contact between the heated bladder and the injection device.

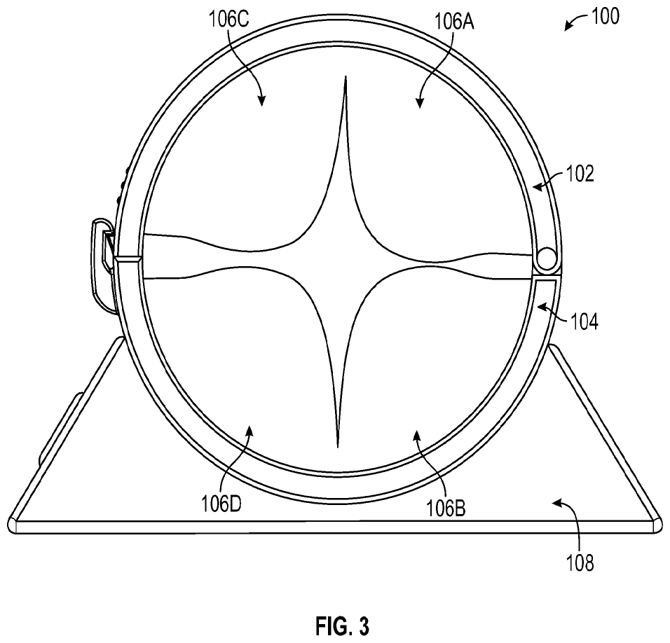

As shown in FIG. 3, when the housing enclosure is in the closed configuration, the plurality of bladders 106A-106D defines a lumen with a star-shaped cross-sectional surface area for contacting and holding an injection device. The illustrated cross-sectional shape is exemplary. The cross-sectional shapes may be a square, circular, oval, or square-shaped with curved sides. In addition, the sizing of the cross-sectional shapes may vary. One exemplary dimension, such as a diameter or a width of the cross-sectional shape, is 8-23 mm. Turning back to FIG. 3, the bladders 106A and 106C are adjacent to an inner surface of the first housing shell 102. For example, the 106A and 106C are symmetrically positioned on the inner surface of the first housing shell 102. The bladders 106B and 106D are adjacent to an inner surface of the second housing shell 104. For example, two bladders 106B and 106D are symmetrically positioned on the inner surface of the second housing shell 104. The bladders may attach to the inner surface of a respective housing shell, or the bladders may be integrated as part of the housing shell.

When an injection device is placed the warming device 100 of FIG. 3, the bladders 106A-106D contact and then conform to the cross-section of the inserted injection device. Because bladders 106A-106D are fluid-filled, they can contact and conform to different injection devices having different device geometries. In this manner, the warming device 100 can accommodate many different injection devices and heat their respective drug formulations via the bladders without compromising drug safety. For example, the different injection devices may be heated without contaminating or overheating the drug formulations.

Because different injection devices may be heated using one device, the warming device 100 lowers the viscosities of different formulations in a more cost-effective and efficient manner. The ability to lower viscosities of different formulations may be even more beneficial for high-concentration formulations, such as a formulation comprising a high concentration of biologic or a high dosage, because the warming device 100 would exponentially lower these formulations' high viscosities and corresponding injection forces, making injection of high-concentration drugs more comfortable. The warming device 100 may also remove aggregations that cause injection problems, including discomfort and pain, by heating the drug formulation and causing the drug viscosity to be more uniformed. Furthermore, because the bladder configuration may surround different injection device cross-sectional shapes, the bladders may evenly contact any injection device around the cross-section, and different drug formulations may be heated more evenly via the surrounding contacts. Additionally, the warming device 100 reduces the wait time for lowering viscosities of different drug formulations. Because the wait time for lowering drug viscosity is reduced, the warming device 100 allows higher-concentration drug formulations, which have a higher viscosity, to be more suitable for use in time-sensitive situations.

Formulations can comprise both small molecules and biologics with various excipients. For instance, certain biologics which can be used with the present embodiments can comprise biologics for treatment of hemophilia disease such as Factor VII, Factor VIII, Factor IX, Factor X, Factor XI, insulin, Betaseron®, and other similar small molecules, biologics and their combinations which are known and used in the art. It is also within the scope of the present embodiments that various volumes can be employed.

Although the exemplary warming device 100 is described with respect to the illustrated bladder configuration, it should be appreciated that the illustrated bladder configuration is exemplary. The warming device 100 may include other bladder configurations for heating drug formulations in different injection devices. As specific examples, an n number of bladders is adjacent to the inner surface of the first housing shell 102, and an m number of bladders is adjacent to the inner surface of the second housing shell 104. Note that n may or may not equal m. The n number of bladders is symmetrically positioned on the inner surface of the first housing shell 102 in some embodiments. The m number of bladders is symmetrically positioned on the inner surface of the second housing shell 104 in some embodiments. Positions of the n bladders on the inner surface of the first housing shell 102 may mirror (about a plane between the first and second housing shells) the positions of the m bladders on the inner surface of the second housing shell 104.

The plurality of bladders may also be arranged according to the shape of the housing enclosure. For example, if the housing enclosure is in a hexagonal prism shape, the warming device may comprise six bladders, each adjacent to a side of the hexagonal cross-section. As another example, if the housing enclosure is in an octagonal prism shape, the warming device may comprise eight bladders, each adjacent to a side of the octagonal cross-section.

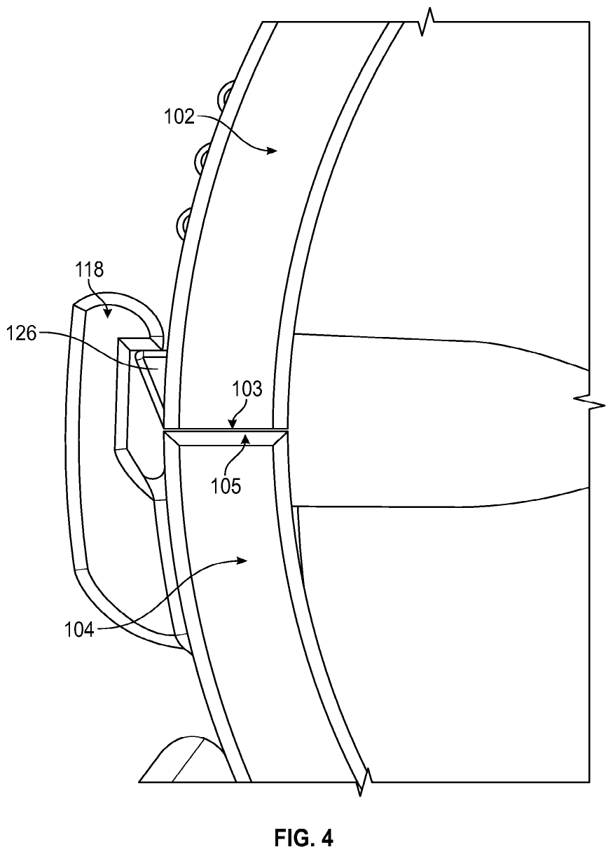

FIG. 4 depicts the side view of warming device 100 in more detail. When the housing enclosure is in the closed configuration, the latch 118 contacts protrusion 126, which is located on the first housing shell 102 above the edge 103. The contact between the latch 118 and the protrusion 126 keeps the first housing shell 102 from rotating and the edges 103 and 105 fixed. A user may cause the latch 118 to de-couple the first housing shell 102 and the second housing shell 104 and open the housing enclosure. For example, the user pulls the latch 118 away from the protrusion 126, and the first housing shell 102 may rotate about hinge 116 to open the housing enclosure. It should be appreciated that the warming device 100 may comprise alternative mechanisms for coupling the first and second housing shells.

By maintaining the coupling between the first housing shell 102 and the second housing shell 104, the latch 118 may advantageously counteract the force of an injection device pushing against the bladders 106A-106D and housing shells, allowing the bladders 106A-106D to stay in contact with the injection device and better heat the injection device via conduction. Without the latch 118, the force of the injection device pushing against the bladders may cause the housing shells to de-couple and thus reduce contact between the bladders and the injection device.

In addition to the components described above, some embodiments of the warming device 100 comprise a sensor or an array of sensors. The sensor or the array of sensors are designed for detecting a temperature of the injection device. For example, the sensor comprises a thermocouple, such as a thermocouple designed to detect 2-50° C. As another example, the array of sensors comprises an array of thermocouples, such as thermocouples designed to detect 2-50° C. The sensor or the array of sensors may be positioned in the fluid in one or more of the bladders 106A-106D. The sensor or the array of sensors may be adjacent to one or more of the bladders 106A-106D. The sensor or the array of sensors may be positioned in a location such that a temperature difference across a bladder may be measured. The sensor allows the warming device 100 to determine whether the drug formulation in the injection device reached a desired temperature or a desired temperature range.

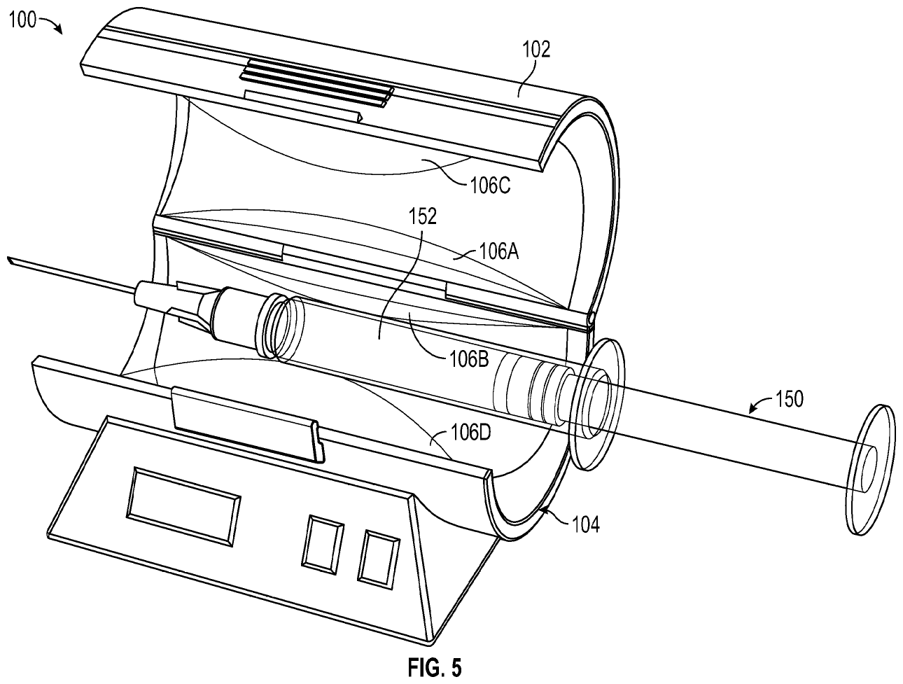

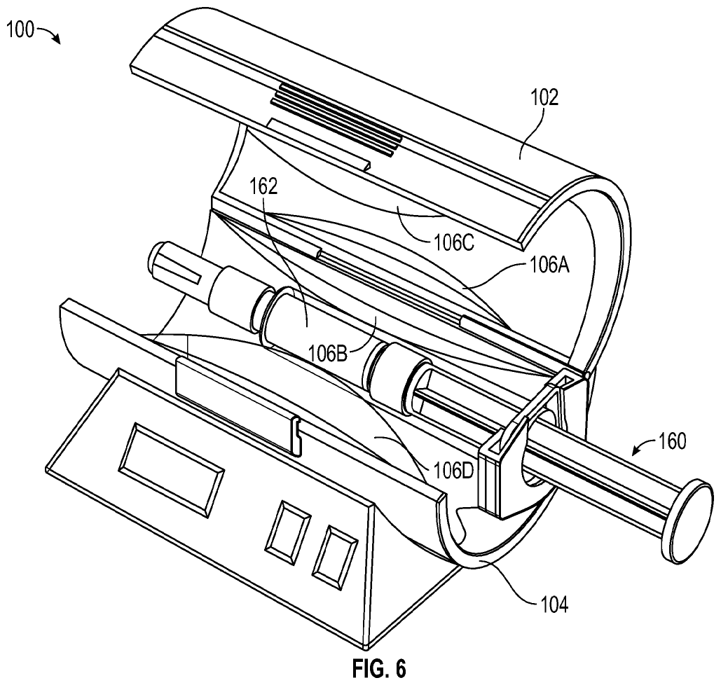

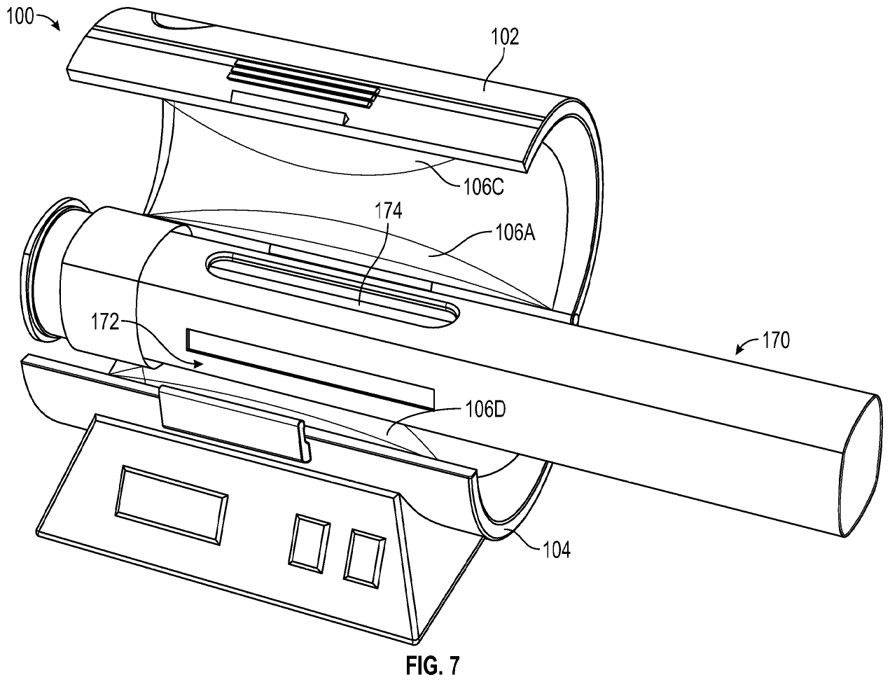

FIGS. 5-7 depict exemplary warming devices 100 when the housing enclosure is in an open configuration and exemplary injection devices, in accordance with some embodiments. As discussed above, the bladders 106A-106D accommodate different injection devices and allow the warming device 100 to heat drug formulations in the different injection devices. For example, the warming device 100 is designed to accommodate the pre-filled syringe 150 of FIG. 5, pre-filled syringe 160 of FIG. 6, and autoinjector 170 of FIG. 7 and heat their respective drug formulations. The drug formulations in the injection devices may comprise a biologic. When the housing enclosure is in an open configuration as shown in FIGS. 5-7, an opening for receiving the different injection devices is provided by opening the first housing shell 102 and the second housing shell 104.

The warming device 100 may comprise a visual indicator (not depicted) for positioning an injection device on the bladders 106B and 106D. For example, the visual indicator is on an inner surface of the second housing shell 104. A user may use the visual indicator to align and orient the injection device to the visual indicator, maximizing the injection device's contact with the bladders. The injection device may comprise a second indicator for alignment with the visual indicator.

The plurality of bladders 106A-106D advantageously accommodates different injection devices, allowing drug formulations in different injection geometries to be heated and lowering the viscosity of the corresponding drug formulations for a more comfortable injection. For example, in FIG. 5, the pre-filled syringe 150 comprises a barrel 152 having a first diameter. The pre-filled syringe 150 may have a volume 1.0 mL-2.25 mL. When the pre-filled syringe 150 is loaded onto the bladders 106B and 106D, the bladders 106B and 106D contact two portions of the barrel 152 and conform to the shape of the barrel 152. The flexible bladders may conform to the shape of the barrel 152 according to the first diameter and the weight of the pre-filled syringe 150. Subsequently, when the housing enclosure is in a closed configuration, the bladders 106A and 106C contact another two portions of the barrel 152 and conform to the barrel 152.

As another example, in FIG. 6, the pre-filled syringe 160 comprises a barrel 162 having a second diameter. The pre-filled syringe 160 may have a volume 1.0 mL-2.25 mL. When the pre-filled syringe 160 is loaded onto the bladders 106B and 106D, the bladders 106B and 106D contact two portions of the barrel 162 and conform to the shape of the barrel 162. The flexible bladders may conform to the shape of the barrel 162 according to the second diameter and the weight of the pre-filled syringe 160. Subsequently, when the housing enclosure is in a closed configuration, the bladders 106A and 106C contact another two portions of the barrel 162 and conform to the barrel 162.

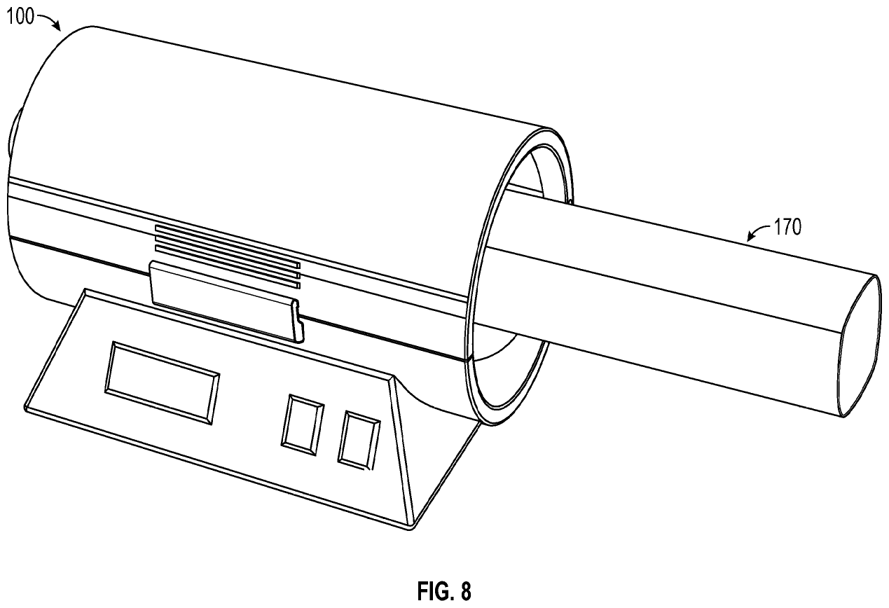

As another example, in FIG. 7, the autoinjector 170 comprises an outer shell 172 having a cross-sectional area. When the autoinjector 170 is loaded onto the bladders 106B and 106D, the bladders 106B and 106D contact two portions of the outer shell 172 and conform to the shape of the outer shell 172. The flexible bladders may conform to the shape of the outer shell 172 according to the cross-sectional area and the weight of the autoinjector 170. Subsequently, when the housing enclosure is in a closed configuration, the bladders 106A and 106C contact another two portions of the outer shell 172 and conform to the outer shell 172, as described with respect to FIG. 8.

As illustrated in FIGS. 5-7, the open-ended structure formed by the housing enclosure advantageously allows the warming device 100 to heat injection devices having different lengths because the open-ended structure allows a portion of an injection device not in contact with the bladders 106A-106D to protrude beyond the warming device 100.

The autoinjector 170 may comprise one or more windows 174. The window 174 is an opening on the outer shell 172 of the autoinjector 170. For example, the width of the window 174 is ¼ to ¾ of the width of a side of the autoinjector 170, and the length of the window 174 is at least ½ of the length of a portion of the autoinjector 170 containing the drug formulation (such as a vial). When the autoinjector 170 is loaded onto the warming device 100 and the housing enclosure is in the closed configuration, as described with respect to FIG. 8, the window 174 allows one or more of the bladders 106A-106D to contact not only the outer shell 172 of the autoinjector 170 but also contact the portion of the autoinjector 170 containing the drug formulation, providing better thermal conduction from the bladders to the drug formulation.

FIG. 8 depicts an exemplary warming device 100 when the housing enclosure is in a closed configuration and the autoinjector 170, in accordance with some embodiments. When the housing enclosure is in the closed configuration, the bladders 106A-106D are designed to contact a portion of the autoinjector 170 and heat the drug formulation in the autoinjector 170. For example, in the closed configuration, the bladders 106A-106D are designed to contact four portions the outer shell 172 (described in FIG. 7), with each bladder contacting a respective portion, and conform to the body 172 according to the cross-sectional area of the outer shell 172. The forces between the bladders and the outer shell 172 at the four portions cause the deformable bladders to conform to the outer shell 172. It should be appreciated that the bladders 106A-106D may conform to other injection device geometries in a similar manner when the housing enclosure is in the closed configuration.

As illustrated by these examples, due to the bladders conforming to the injection device geometry, the bladders 106A-106D contact and conform to different injection devices having different device geometries, allowing different formulations in different injection devices to be heated via the bladders. In this manner, the warming device 100 can accommodate many different injection devices and heat their respective drug formulations via the bladders without compromising drug safety. For example, the different injection devices may be heated without contaminating or overheating the drug formulations.

Because different injection devices may be heated using one device, the warming device 100 lowers the viscosity of different formulations in a more cost-effective and efficient manner. The ability to lower viscosities of different formulations may be even more beneficial for high-concentration formulations, such as a formulation comprising a high concentration of biologic or a high dosage, because the warming device 100 would exponentially lower these formulations' high viscosities and corresponding injection forces, making injection of high-concentration drugs more comfortable. The warming device 100 may also remove aggregations that cause injection problems, including discomfort and pain, by heating the drug formulation and causing the drug viscosity to be more uniformed. Furthermore, because the bladder configuration may surround different injection device cross-sections, the bladders may evenly contact the injection device around the cross-section, and different drug formulations may be heated more evenly via the contacts.

Although FIGS. 5-8 are described with respect to pre-filled syringes and an autoinjector, it should be appreciated that the warming device 100 may receive and heat other injection devices to achieve the benefits described herein. For example, the injection device may be an ampoule having a volume up to 20 mL or a cartridge having a volume up to 5 mL.

The present invention may comprise a kit having the warming device 100 and one or more injection devices (for example, as described with respect to FIGS. 5-8). The warming device 100 is designed to heat the injection device of the kit, achieving the benefits described herein. Because the warming device 100 lowers the viscosities of different formulations in different injection devices in a more cost-effective and efficient manner, injection devices containing different formulations having different viscosities may be included in the kit with the same warming device. Some embodiments of the kit may have additional elements. For example, the kit may have a swab and antiseptic solution to facilitate injection of the heated drug formulation into the patient and an optional instruction set.

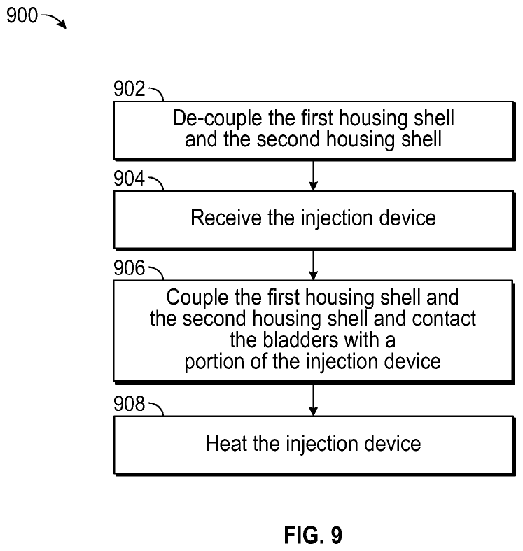

FIG. 9 depicts an exemplary method 900 of heating a drug formulation, in accordance with some embodiments. The method 900 may be performed with warming device 100. It should be appreciated that the steps of the method 900 leverage the features and advantages of warming device 100. Although the method 900 is illustrated as including the described steps, it should be appreciated that a different order of steps, additional steps, or fewer steps may be included without departing from the scope of the disclosure.

The method 900 comprises de-coupling an edge of the first housing shell of the warming device and an edge of the second housing shell of the warming device and providing an opening (step 902). For example, as described in FIGS. 2 and 5-7, the edge 103 of the first housing shell 102 and the edge 105 of the second housing shell 104 are de-coupled when the housing enclosure of warming device 100 is in an open configuration, and an opening is provided for receiving an injection device.

The method 900 comprises receiving, via the opening, the injection device (step 904). For example, the opening is provided by the warming device 100 when the edge 103 of the first housing shell 102 and the edge 105 of the second housing shell 104 are de-coupled. An injection device (for example, as described with respect to FIGS. 5-7) is received by the warming device 100 via the opening.

The method 900 comprises coupling the edge of the first housing shell and the edge of the second housing shell and contacting the warming device's plurality of bladders with a portion of the injection device (step 906). For example, the edge 103 of the first housing shell 102 and the edge 105 of the second housing shell 104 are coupled when the housing enclosure of warming device 100 is in a closed configuration. When the housing enclosure is in the closed configuration, the bladders 106A-106D contact a portion of the injection device (for example, as described with respect to FIGS. 3 and 8).

Coupling the edge of the first housing shell and the edge of the second housing shell may further comprise latching the edge of the first housing shell and the edge of the second housing shell. For example, as described with respect to FIGS. 1 and 4, the latch 118 keeps the edge 103 of the first housing shell 102 and the edge 105 of the second housing shell 104 coupled.

The method 900 may comprise causing the plurality of bladders to deform to conform to the portion of the injection device. For example, when the housing enclosure is in the closed configuration, the inflated bladders 106A-106D contact and conform to the geometry of the portion of the injection device (for example, barrel 152, barrel 162, or outer shell 172). In this manner, performing method 900 allows the bladders to accommodate many different injection devices and respective drug formulations may be heated via the bladders without compromising drug safety. For example, the different injection devices may be heated without contaminating or overheating the drug formulations.

The method 900 comprises heating, via the plurality of bladders, the injection device (step 908). For example, when the housing enclosure is in the closed configuration and the injection device is received by the warming device 100, the heating element of the warming device 100 provides heat to the drug formulation of the injection device through the contact between the bladders 106A-106D and the portion of the injection device via thermal conduction. The plurality of bladders may comprise a fluid, and heating, via the plurality of bladders, the injection device comprises heating the fluid at one atmosphere to about 21-22.2° C. within five seconds to 10 minutes from storage temperature. The solution may be heated by the heating element via thermal conduction, and the storage temperature may be 2-8° C.

As discussed above, the viscosity of a drug formulation is based on temperature and/or concentration. For example, the concentration of the drug formulation is 50-150 mg/mL, and the warming device 100 may heat the drug formulation to room temperature and lower the viscosity of the drug formulation to 1-30 centipoise (cP).

Because different injection devices may be heated via the plurality of bladders, the method 900 allows the viscosities of different formulations to be lowered in a more cost-effective and efficient manner. The ability to lower viscosities of different formulations may be even more beneficial for high-concentration formulations, such as a formulation comprising a high concentration of biologic or a high dosage, because performing method 900 would exponentially lower these formulations' high viscosities and corresponding injection forces, making injection of high-concentration drugs more comfortable. Performing method 900 may also remove aggregations that cause injection problems, including discomfort and pain, by heating the drug formulation and causing the drug viscosity to be more uniformed. Furthermore, because the bladder configuration may surround different injection device cross-sectional shapes, the bladders may evenly contact any injection device around the cross-section, and different drug formulations may be heated more evenly via the surrounding contacts. Additionally, performing method 900 reduces the wait time for lowering viscosities of different drug formulations. Because the wait time for lowering drug viscosity is reduced, performing method 900 allows higher-concentration drug formulations, which have a higher viscosity, to be more suitable for use in time-sensitive situations.

As discussed earlier, some embodiments of the warming device 100 comprise a sensor for detecting a temperature of the injection device. The sensor may be designed for providing temperature feedback. The warming device 100 may further comprise a controller or a processor for determining whether the drug formulation in the injection device reached a desired temperature or a desired temperature range based on an output of the sensor. For example, the feedback may be used to determine whether the drug formulation in the autoinjector 170 reached a desired temperature or a desired temperature range.

The method 900 may comprise determining, based on an output of a sensor of the warming device, whether the temperature of the injection device has reached a desired temperature or a desired temperature range. For example, based on the output of the sensor, the warming device 100 determines whether the temperature of the drug formulation in the injector 170 in the housing enclosure has reached room temperature.

The method 900 may comprise ceasing heating the drug formulation in accordance with a determination that the temperature of the injection device has reached the desired temperature or the desired temperature range. For example, based on the output of the sensor, the warming device 100 determines that temperature of the drug formulation in the autoinjector 170 in the housing enclosure has reached room temperature. In accordance with this determination, the warming device 100 ceases heating the drug formulation in autoinjector 170, for example, by deactivating the heating element.

The method 900 may comprise maintaining the temperature of the injection device at the desired temperature or the desired temperature range. For example, after the warming device 100 ceases heating the drug formulation in the injection device (in accordance with the determination that the temperature of the injection device has reached the desired temperature or the desired temperature range), the warming device 100 maintains the injection device at the desired temperature or the desired temperature range while the autoinjector 170 is in the warming device 100, allowing the drug formulation to retain its described benefits at the desired temperature or desired temperature range until injection. The desired temperature or the desired temperature range may be maintained, for example, up to 30 minutes. The warming device 100 may maintain the temperature of a drug formulation until the housing enclosure is opened. The warming device 100 may maintain the temperature of a drug formulation in response to receiving an input, such as a selection of the heat activation button 110 or a different button.

The method 900 may comprise continuing heating the drug formulation in accordance with a determination that the temperature of the injection device has not reached the desired temperature or the desired temperature range. For example, based on the output of the sensor, the warming device 100 determines that temperature of the injection device has not reached room temperature while the autoinjector 170 is in the warming device 100. In accordance with this determination, the warming device 100 continues heating the drug formulation in the autoinjector 170.

The method 900 may comprise determining, based on an output of the sensor, that the temperature of the bladders is decreasing. In accordance with a determination that the temperature of the bladders is decreasing, the bladders 106A-106D are heated again, via the heating element, to keep the bladders at a temperature range for heating a drug formulation.

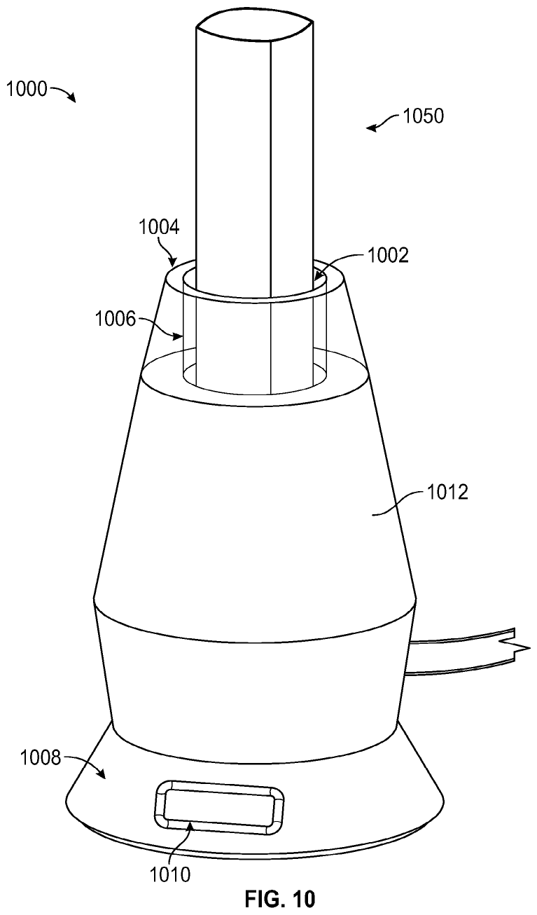

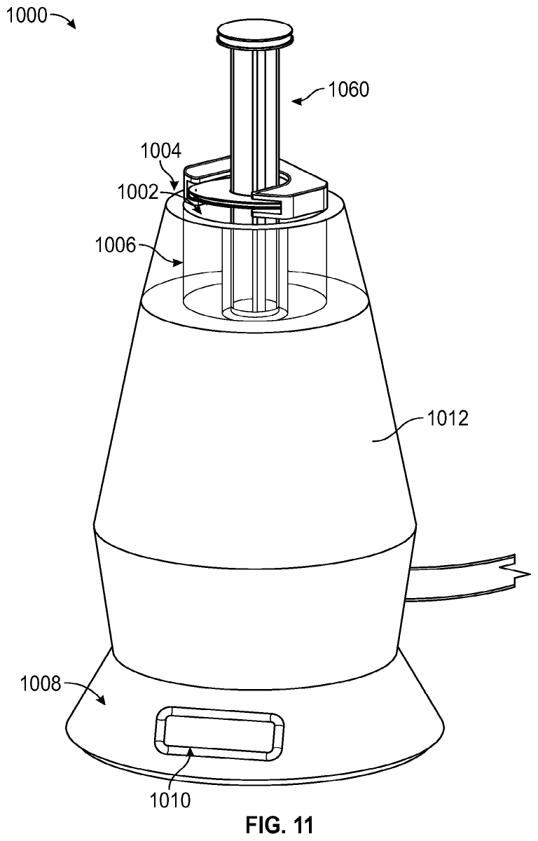

FIGS. 10-11 depict exemplary warming devices 1000 and exemplary injection devices, in accordance with some embodiments. The warming device 1000 comprises a heating chamber 1002, an opening 1004 to the heating chamber, a temperature indicator light 1006, a base 1008, a heat activation button 1010, and a housing 1012. An injection device, such as autoinjector 1050 or pre-filled syringe 1060, may be inserted into the heating chamber 1002, and the heating chamber 1002 heats the injection device.

The base 1008 is located at a bottom of the warming device 1000, and the heat activation button 1010 is disposed on the base 1008. The housing 1012 is attached above the base 1008 along a direction of injection device insertion. The temperature indicator light 1006 may be a transparent lighting component attached above the housing 1012. The opening 1004 is located at a top of the warming device 1000, on a side of the warming device 1000 opposing the base 1008. The base 1008, the housing 1012, the temperature indicator light 1006, and the opening 1004 may have semi-conical shapes, as illustrated. The diameters of the cross-section of the base 1008 and the housing 1012 at their interface may be the same. The diameters of the cross-section of the housing 1012 and temperature indicator light 1006 at their interface may be the same.

The heating chamber 1002 is located inside the housing 1012 and comprises an enclosure that is designed to surround a portion of the inserted injection device and heat the drug formulation in the injection device to a desired temperature or a desired temperature range. The warming device 1000 may comprise a heating element for providing heat to the heating chamber 1002. For example, the heating element is positioned adjacent to walls of the enclosure of the heating chamber 1002 and transfers heat, via air, to the portion of the injection device in the enclosure. The heating element may be positioned to align with a location of the drug formulation in the injection device, such as a barrel of a pre-filled syringe or a window of an autoinjector. The heating element may comprise an electrical heating element such as heating coils, a heating ribbon, or a heating mat.

As another example, the heating element may contact a plurality of bladders (not depicted), such as bladders 106A-106D, and the bladders may be positioned adjacent to walls of the enclosure of the heating chamber 1002. Through conduction, the heating element provides heat to the bladders, and the bladders contact the portion of the injection device in the heating chamber 1002 and heat the injection device.

The opening 1004 is configurable to accept injection devices having different cross-section shapes. For example, the width of the opening 1004 may be adjustable for conforming to a cross-section of an injection device. For example, the width of the opening 1004 may be adjustable to conform to the width of autoinjector 1050 or pre-filled syringe 1060. The opening 1004 may be narrower than a flange of pre-filled syringe 1060, allowing the flange to rest on the opening 1004 while the pre-filled syringe is being heated. Allowing the flange to rest on the opening 1004 may prevent a portion of the injection device from entering the heating chamber 1002 and prevent unnecessary heating of this portion (such as a plunger of a syringe that does not need to be heated).

Because different injection devices may be inserted into the warming device 1000, the heating chamber 1002 and opening 1004 advantageously allow drug formulations in different injection devices having different geometries to be heated and the viscosities of the different formulations to be lowered using one device.

The temperature indicator light 1006 indicates a state of the heating chamber 1002. For example, when the heating chamber 1002 is heating an injection device, the temperature indicator light 1006 illuminates a first color, for example, red. When the heating chamber 1002 finishes heating an injection device (for example, when the drug formulation in the injection device reaches a desired temperature), the temperature indicator light 1006 illuminates a second color, for example, green. When the heating chamber 1002 is not heating an injection device, the temperature indicator light does not illuminate.

The base 1008 provides stability for the warming device 1000 when the device is placed on a surface. The base 1008 may house electronic components for controlling the warming device 1000. In response to receiving a selection of the heat activation button 1010, the warming device 1000 is designed to heat an inserted injection device via the heating chamber 1002, as described above. For example, in response to a user pushing the heat activation button 1010, the warming device 1000 heats an inserted injection device via the heating chamber 1002.

Although warming device 1000 is described as comprising one button and one light, it should be appreciated that the warming device 1000 may comprises additional, fewer, or different user interface elements. For example, the warming device 1000 may comprise an interface for temperature selection. The interface may be designed for setting a desired temperature or for selecting a desired temperature. As another example, the warming device 1000 may comprise an interface for receiving information associated with the drug formulation or the injection device to be heated. The received information may be used to determine heating time.

The warming device 1000 may comprise a sensor (not shown) for detecting a temperature of the injection device. For example, the warming device 1000 comprises a thermocouple or an array of thermocouples designed for providing temperature feedback. The temperature feedback may be used to determine whether the drug formulation in the injection device reached a desired temperature or a desired temperature range.

The warming device 1000 is electrically powered. For example, the warming device 1000 comprises a power cord for receiving power from a power outlet. As another example, the warming device 1000 is powered by one or more batteries. The one or more batteries may be rechargeable via the power from the power cord.

Some embodiments of a kit comprise the warming device 1000 and one or more injection devices. The warming device 1000 is designed to heat the injection device of the kit, achieving the benefits described herein.

According to some embodiments, a device for heating a drug formulation in an injection device comprises: a housing enclosure comprising: a first housing shell having an inner surface, a second housing shell having an inner surface. When the housing enclosure is in an open configuration, an edge of the first housing shell and an edge of the second housing shell are de-coupled to provide an opening for receiving the injection device. The device further comprises: a plurality of bladders comprising: a first bladder adjacent to the inner surface of the first housing shell, and a second bladder adjacent to the inner surface of the second housing shell; and a heating element designed to provide heat to the plurality of bladders for heating the drug formulation. When the housing enclosure is in a closed configuration: the edge of the first housing shell and the edge of the second housing shell couple and form an open-ended structure, and the bladders contact a portion of the injection device for heating the drug formulation in the injection device.

According to some embodiments, the plurality of bladders comprises enclosures for containing a fluid.

According to some embodiments, the device further comprises a sensor for detecting a temperature of the injection device.

According to some embodiments, the sensor comprises a thermocouple.

According to some embodiments, when the housing enclosure is in the closed configuration, the plurality of bladders defines a lumen with a star-shaped cross-sectional surface area for contacting and holding the injection device.

According to some embodiments, the plurality of bladders is inflated and deformable to conform to the portion of the injection device.

According to some embodiments, the plurality of bladders comprises silicone, nylon, latex, rubber, an elastomer material, or combination thereof.

According to some embodiments, the injection device comprises an autoinjector.

According to some embodiments, the injection device comprises a pre-filled syringe.

According to some embodiments, the device further comprises a base attached to the housing enclosure.

According to some embodiments, the device comprises a latch for coupling the edge of the first housing shell and the edge of the second housing shell.

According to some embodiments, the drug formulation comprises a biologic.

According to some embodiments, a portion of the injection device not in contact with the plurality of bladders protrudes from the open-ended structure.

According to some embodiments, a kit comprises: an injection device and a device for heating a drug formulation in the injection device, comprising: a housing enclosure comprising: a first housing shell having an inner surface, a second housing shell having an inner surface. When the housing enclosure is in an open configuration, an edge of the first housing shell and an edge of the second housing shell are de-coupled to provide an opening for receiving the injection device. The device further comprises: a plurality of bladders comprising: a first bladder adjacent to the inner surface of the first housing shell, and a second bladder adjacent to the inner surface of the second housing shell; and a heating element designed to provide heat to the plurality of bladders for heating the drug formulation. When the housing enclosure is in a closed configuration: the edge of the first housing shell and the edge of the second housing shell couple and form an open-ended structure, and the bladders contact a portion of the injection device for heating the drug formulation in the injection device.

According to some embodiments, a method for heating a drug formulation in an injection device using a warming device comprises: de-coupling an edge of a first housing shell of the warming device and an edge of a second housing shell of the warming device and providing an opening; receiving, via the opening, the injection device; coupling the edge of the first housing shell and the edge of the second housing shell and contacting a plurality of bladders of the warming device with a portion of the injection device; and heating, via the plurality of bladders, the injection device.

According to some embodiments, heating, via the plurality of bladders, the injection device comprises heating the bladders at one atmosphere to about 21-22.2° C. within five seconds to 10 minutes from storage temperature.

According to some embodiments, the method further comprises: determining, based on an output of a sensor of the warming device, whether the temperature of the injection device has reached a desired temperature or a desired temperature range; in accordance with a determination that the temperature of the injection device has reached the desired temperature or the desired temperature range, ceasing heating the drug formulation; and in accordance with a determination that the temperature of the injection device has not reached the desired temperature or the desired temperature range, continuing heating the drug formulation.

According to some embodiments, the method further comprises maintaining the temperature of the injection device at a desired temperature or a desired temperature range.

According to some embodiments, the plurality of bladders is inflated, and the method further comprises causing the plurality of bladders to conform to the portion of the injection device.

According to some embodiments, coupling the edge of the first housing shell and the edge of the second housing shell further comprises latching the first housing shell and the edge of the second housing shell.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are enabled to best utilize the techniques and various embodiments with various modifications as are suited to the particular use contemplated.

What is claimed is:

1. A device for heating a drug formulation in an injection device, comprising:
a housing enclosure comprising:
a first housing shell having an inner surface,
a second housing shell having an inner surface,
wherein, when the housing enclosure is in an open configuration, an edge of the first housing shell and an edge of the second housing shell are de-coupled to provide an opening for receiving the injection device;
a plurality of bladders, comprising:
a first bladder adjacent to the inner surface of the first housing shell, and a second bladder adjacent to the inner surface of the second housing shell; and
a heating element designed to provide heat to the plurality of bladders for heating the drug formulation;
wherein, when the housing enclosure is in a closed configuration:
the edge of the first housing shell and the edge of the second housing shell couple and form an open-ended structure, and
the bladders contact a portion of the injection device for heating the drug formulation in the injection device.

2. The device of claim 1, wherein the plurality of bladders comprises enclosures for containing a fluid.

3. The device of claim 1, further comprising a sensor for detecting a temperature of the injection device.

4. The device of claim 1, wherein, when the housing enclosure is in the closed configuration, the plurality of bladders defines a lumen with a star-shaped cross-sectional surface area for contacting and holding the injection device.

5. The device of claim 1, wherein the plurality of bladders is inflated and deformable to conform to the portion of the injection device.

6. The device of claim 1, wherein the injection device comprises an autoinjector.

7. The device of claim 1, wherein the injection device comprises a pre-filled syringe.

8. The device of claim 1, further comprising a base attached to the housing enclosure.

9. The device of claim 1, further comprising a latch for coupling the edge of the first housing shell and the edge of the second housing shell.

10. A method for heating a drug formulation in an injection device using a warming device, comprising:
de-coupling an edge of a first housing shell of the warming device and an edge of a second housing shell of the warming device and providing an opening;
receiving, via the opening, the injection device;
coupling the edge of the first housing shell and the edge of the second housing shell and contacting a plurality of bladders of the warming device with a portion of the injection device; and
heating, via the plurality of bladders, the injection device.

11. The method of claim 10, wherein heating, via the plurality of bladders, the injection device comprises heating the bladders at one atmosphere to about 21-22.2° C. within five seconds to 10 minutes from storage temperature.

12. The method of claim 10, further comprising:
determining, based on an output of a sensor of the warming device, whether the temperature of the injection device has reached a desired temperature or a desired temperature range;
in accordance with a determination that the temperature of the injection device has reached the desired temperature or the desired temperature range, ceasing heating the drug formulation; and
in accordance with a determination that the temperature of the injection device has not reached the desired temperature or the desired temperature range, continuing heating the drug formulation.

13. The method of claim 10, further comprising maintaining the temperature of the injection device at a desired temperature or a desired temperature range.

14. The method of claim 10, wherein the plurality of bladders are inflated, and the method further comprises causing the plurality of bladders to conform to the portion of the injection device.

15. The method of claim 10, wherein coupling the edge of the first housing shell and the edge of the second housing shell further comprises latching the first housing shell and the edge of the second housing shell.
